(19) United States
(12) Patent Application Publication
(10) Pub. No.: US 2016/0128696 A1
(43) Pub. Date: May 12, 2016

(54) INTRA-OPERATIVE PRESSURE CUFF TO REDUCE ORGAN BLEEDING

(71) Applicant: Teresa COTTERALL-LAGANA, New Rochelle, NY (US)

(72) Inventor: Teresa COTTERALL-LAGANA, New Rochelle, NY (US)

(21) Appl. No.: 14/898,145

(22) PCT Filed: Jun. 13, 2014
(86) PCT No.: PCT/IB14/62198

§ 371 Date: Dec. 14, 2015

Related U.S. Application Data
(60) Provisional application No. 61/834,678, filed on Jun. 13, 2013.

(51) Int. Cl. A61B 17/12

(52) U.S. Cl. CPC A61B 17/12; A61B 2017/12004

(57) ABSTRACT

An embodiment of the invention relates to an apparatus comprising: a flexible sleeve configured to envelop at least a portion of a uterus; and an adjustable pressure applicator configured to provide pressure on a uterus of a patient. According to an embodiment of the invention, the pressure applicator is a bladder adapted to receive and dispel a fluid. Embodiments of the invention relate to use of the apparatus to control post partum hemorrhage, in particular, after caesarean section delivery.

### RELATED APPLICATIONS

The present application claims the benefit under 35 U.S.C. 119(e) of U.S. Provisional Application 61/834,678 filed on Jun. 13, 2013, the disclosure of which is incorporated herein by reference.

### TECHNICAL FIELD

Embodiments of the present invention relate to devices for reduction of organ bleeding, in particular, bleeding of the uterus.

### BACKGROUND

After delivery of a neonate in vaginal childbirth or caesarean section childbirth, a woman's uterus, in normal conditions, tends to bleed as a result of the detachment of the placenta from the uterus. In most births, the uterus contracts shortly after childbirth, slowing the blood flow from the uterus. Abnormal bleeding of the uterus after childbirth is known as postpartum hemorrhage (PPH). PPH is typically present in situations in which the myometrium (uterine muscle) does not contract the uterus, allowing for profuse bleeding to continue after childbirth. PPH is a relatively common symptom of childbirth but is more common after caesarean section delivery than after vaginal delivery. In some instances, PPH results from placenta accreta, a condition in which the placenta is deeply attached to the myometrium, resulting in severe bleeding at the time of the removal of the placenta from the uterus.

If left uncontrolled, PPH can lead to rapid blood loss, warranting blood transfusions. In extreme situations, PPH may lead to disseminated intravascular coagulation (DIC) which may, in turn, lead to death.

Pharmacological interventions to control PPH include administration of oxytocin to contract the uterus. Mechanical interventions which can be used in early stages of PPH include manual massage of the fundus of the uterus and application of sutures, known as B-Lynch sutures, to slow the bleeding. In extreme cases, PPH is treated with hysterectomy.

### SUMMARY

An aspect of an embodiment of the invention relates to an apparatus, known as an intra-operative pressure cuff (IOPC) for the control and treatment of PPH. The IOPC, according to an embodiment of the invention, may comprise a flexible sleeve which may be used to wrap around at least a portion of the uterus, at least one closure to secure the sleeve around the uterus and a pressure applicator to apply pressure on the uterus.

According to an embodiment of the invention, the IOPC is easily and quickly adjustable and quickly secured to an enlarged or engorged uterus during an operative procedure.

According to an embodiment of the invention, the pressure applicator is a bladder which can be filled with a fluid. The fluid may be a liquid or a gas. The gas may be air. The bladder may be positioned adjacent to the uterus or surrounding the uterus and configured to apply pressure selectively to the fundus of the uterus without applying substantial pressure to the cervix, thereby allowing blood to exit the uterus via the cervix.

According to an embodiment of the invention, the sleeve may be configured so that in the closed position, the fallopian tubes and vagina of a patient are located outside of the sleeve.

Devices according to embodiments of the invention may reduce amount of blood loss during PPH, in particular in patients in whom PPH results from a caesarean section delivery, thereby reducing and/or eliminating need for transfusions of blood and/or blood products to a patient.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to figures attached hereto that are listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1A shows an IOPC apparatus according to an embodiment of the invention, during application to a uterus during an operative procedure;

FIG. 1B shows an IOPC apparatus in operation after application to and enveloping a uterus according to an embodiment of the invention; and

FIGS. 2A-2C show pressure applicators which may be used in IOPC apparatuses according to embodiments of the invention.

### DETAILED DESCRIPTION

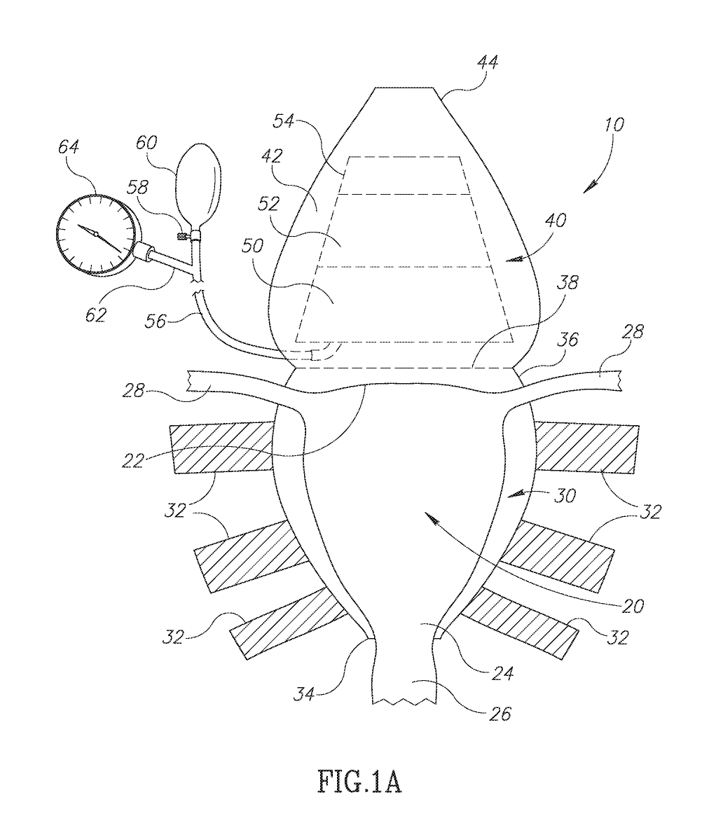

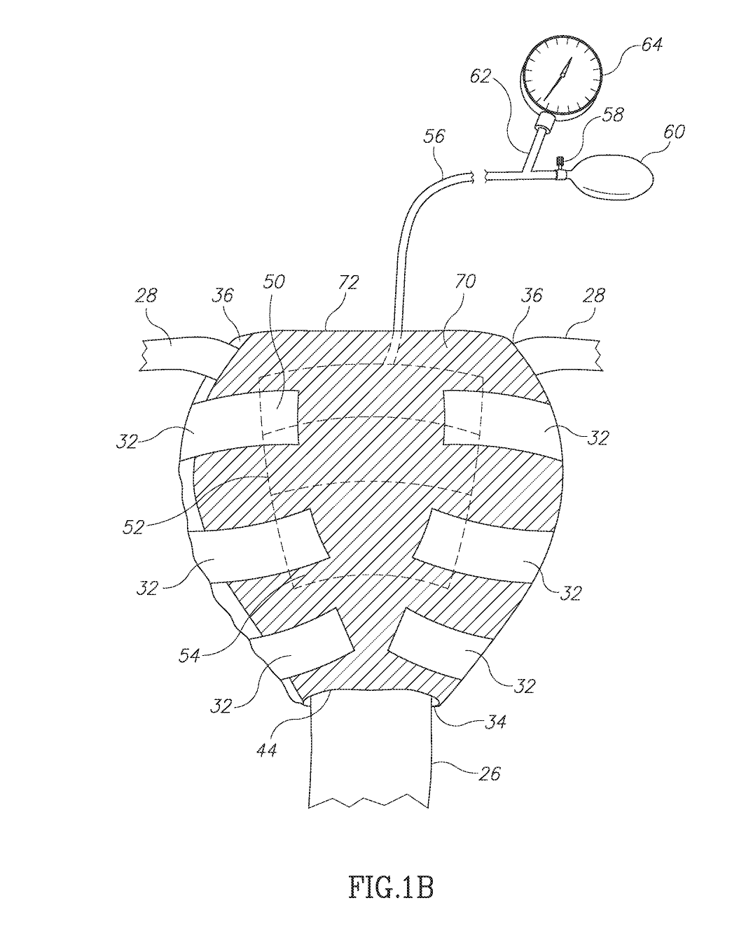

An IOPC is described in detail with reference to FIGS. 1A and 1B.

FIG. 1A shows an IOPC 10 according to embodiments of the invention. IOPC 10 is shown during application to a human uterus 20. Uterus 20 comprises a fundus 22, a cervix 24 and fallopian tubes 28. A vagina 26 is shown. IOPC 10 comprises a rear section 30, which is shown positioned posterior relative to uterus 20. IOPC 10 further comprises a front section 40 configured to fold over at fold 38 and cover the uterus at a position anterior to uterus 20, as shown in FIG. 1B. Returning to FIG. 1A, IOPC further comprises tabs 32 configured to close and secure front section 40 to rear section 30. IOPC 10 comprises openings 36 through which fallopian tubes 28 may be positioned.

IOPC 10 inner layer (designated inner layer as it presents towards the cavity of the device, towards the uterus, when the device is in operation) may comprise a fabric layer 42 along the internal surface of both front section 40 and rear section 30. The fabric of layer 42 may be either as woven or non-woven fabric. The fabric may be either synthetic or natural. A single piece of fabric, without seams presenting on the internal surface may be used as fabric layer 42. Rear section 30 may further comprise cervical opening region 34 and front section 40 may comprise a corresponding cervical opening region 44.

IOPC 10 further comprises a pressure applicator comprising bladders 50, 52 and 54.

Bladder 50 may be flow-connected to an air tube 56. Air tube 56 may be connected to a pump 60 via a release valve 58. Bladder 50 may be connected to a pressure gauge 64 via air tube 62.

IOPC 10 may comprise an outer layer (designated outer layer, as it presents towards the outer surface when IOPC is in place, encompassing a uterus) comprising durable fabric. The fabric may be natural or synthetic, woven or non-woven. According to an embodiment of the invention, the fabric is bendable. The outer layer may comprise a hook and loop material.

Fabric layer 42 can be made from sterile, low-lint or lint-free fabric. Fabric layer 42 may be treated by administering sterile saline solution before use of IOPC 10. Fabric layer 42, according to an embodiment of the invention, is an absorbent, non-stick fabric, which may contact an external surface of a uterus and will not stick to the outer surface after application of pressure to stop postpartum hemorrhage. According to an embodiment of the invention, fabric layer 42 may comprise cotton.

Tabs 32 may comprise either hooks or loops of a fabric hook and loop fastener. The other, matching side of the hook and loop fastener is shown in FIG. 1B, as fastener 70. For example, tabs 32 may comprise hook fabric and fastener 70 may comprise corresponding loop fabric. The hook and loop fastener may comprise Velcro. Tabs 32 or fastener 70 may comprise nylon, polyester, Teflon or combinations thereof.

According to an embodiment of the invention, tabs 32 and corresponding fastener 70 comprise hooks and corresponding eyes, male and corresponding female snap-elements, buttons and corresponding button-holes or zippers.

Bladder 50, 52 and 54 may be made from a fluid-tight material and may be have a relatively flat shape before filling with fluid, and may expand upon filling with fluid. The bladders may be configured to withstand pressure upon filling with fluid such as air, a gas, or a liquid such as water. Upon application of IOPC 10 to the uterus, bladders may surround and assume the shape of the uterus.

According to an embodiment of the invention, bladders 50, 52 and 54 are positioned between IOPC 10 outer and inner layer.

Reference is now made to FIG. 1B. FIG. 1B shows an IOPC apparatus 10 in operation after application to and enclosure around a uterus according to an embodiment of the invention. From a position shown in FIG. 1A, front section 40 is folded at fold 38 and front section 40 is positioned anterior to uterus 20. Tabs 32 are closed, securing front section 40 to rear section 30, thereby securing uterus 20 within a sleeve formed between front section 40 and rear section 30. Tabs 32 are closed by securely applying to corresponding fastener 70.

Upon closing, front section 40 conforms to anterior side of uterus 20 and rear section 30 conforms to posterior side of uterus 20.

According to an embodiment of the invention, fastener 70 covers a majority of the outer surface of front section 40. An operator of IOPC 10 closing tabs 32 on fastener 70 may easily and quickly fasten front section 40 to rear section 30 as a result of the relatively large amount of the outer surface of front section 40 being covered by fastener 70.

Closed IOPC 10, as shown in FIG. 1B has 3 openings: two openings 36 to allow closure of

IOPC 10 without applying substantial pressure to fallopian tubes 28, and an opening defined by cervical opening region 34 and cervical opening region 44.

As shown in FIG. 1A, the pressure applicator comprising bladders 50, 52, 54 is positioned within front section 40. According to embodiments of the invention, pressure applicator is positioned within rear section 30. According to embodiments of the invention, a pressure applicator is positioned within both front section 40 and rear section 30.

According to an embodiment of the invention, bladders 50, 52 and 54 are all flow connected via channels which convey fluid between the bladders.

According to an embodiment of the invention, thickness and/or flexibility of bladders 50, 52 and 54 differ from each other. For example, bladder 50 may be formed from elastic material having higher elasticity or less thickness than bladder 52, and bladder 52 may be formed from elastic material having higher elasticity or less thickness than bladder 54. As a result, upon securing front section 40 to rear section 30 and enclosing uterus 20, filling of bladder 50 via pump 60 and air tube 56 may cause thickness of bladder 50 to increase to a greater extent than the thickness of bladder 52 and bladder 54. As a result, regions of the uterus close to fundus 22 may have more pressure applied to them by inflation of bladder 50 than regions of the uterus close to cervix 24 which have pressure applied to them by inflation of bladders 52 and bladder 54.

According to an embodiment of the invention, IOPC 10 comprises one bladder, two bladders, three bladders, four bladders, five bladders or six bladders. According to an embodiment of the invention, a single bladder may be made of different types or thicknesses of materials to have varying levels of elasticity throughout the bladder. Sections of bladder corresponding to and closest to fundus 22 may have increased elasticity relative to sections of bladder closest to cervix 24. Variation of bladder elasticity may, upon application of IOPC 10 apply pressure selectively to the fundus of the uterus without applying substantial pressure to the cervix, or without applying substantial pressure to uterine regions near the cervix, thereby allowing blood to exit the uterus via the cervix.

According to an embodiment of the invention, bladders 50, 52 and 54 are not flow connected to each other and can be inflated or deflated independently of each other. An operator of IOPC 10 may manually control pressure applied to each bladder to apply pressure selectively to the fundus of the uterus without applying substantial pressure to the cervix, or without applying substantial pressure to uterine regions near the cervix. For example, an operator of IOPC 10 may, upon initial application of IOPC 10 to a uterus, apply pressure to fundal and cervical regions for a designated time, then release pressure to cervical region to allow blood to flow from the cervical region, out through the vagina.

After securing IOPC 10 to enclose uterus 20, the pressure applicator may be engaged to apply pressure to the uterus. According to an embodiment of the invention, the pressure applicator comprises a bladder 50 which is filled by pumping air using pump 60. According to an embodiment of the invention, air pump 60 comprises a one-way valve which introduces air into bladder 50 via air tube 56. Pressure may be released using release valve 58 if too much pressure is applied to uterus 20, or if PPH has slowed or stopped.

An operator may monitor the amount of pressure applied to the uterus by using gauge 64.

Multiple gauges may be used in IOPC 10 to measure pressure in various areas of the uterus. Optionally, pressure in various bladders may be adjusted accordingly.

According to an embodiment of the invention, another source of compressed fluid, such as an air tank, may replace air pump 60. According to an embodiment of the invention, a bladder may be filled with a liquid, for example water, or another gas such as nitrogen, oxygen or carbon dioxide.

After uterine blood flow slows or stops, an operator may release pressure applied by pressure applicator, for example through release valve 58. After release of pressure, if blood flow remains slowed or stopped, an operator may remove tabs 32 from fastener 70. IOPC 10 may then be removed from the patient suffering from PPH.

IOPC 10 may further comprise a controller to automate or partially automate pressure application corresponding to blood flow from the uterus. IOPC 10 may further comprise a blood flow sensor such as a Doppler flow sensor which may be linked to the controller. IOPC 10 may further comprise a pressure valve linked to the controller to control pressure of fluid in bladder 50. A blood flow sensor may be configured to measure blood flow from the cervix to the vagina or from the vagina out of the body of a patient. An operator of IOPC 10 may configure controller of IOPC 10 to apply pressure above a certain threshold to a region of the uterus if blood flow, as measured by the blood flow sensor, is above a certain threshold value. Pressure may be applied, for example, by controller opening a valve to allow pressurized fluid to flow into a bladder. Once blood flow, as sensed by the blood flow sensor, drops below a certain value, the controller may adjust the pressure applied to the region of the uterus, for example, by reducing pressure using a valve.

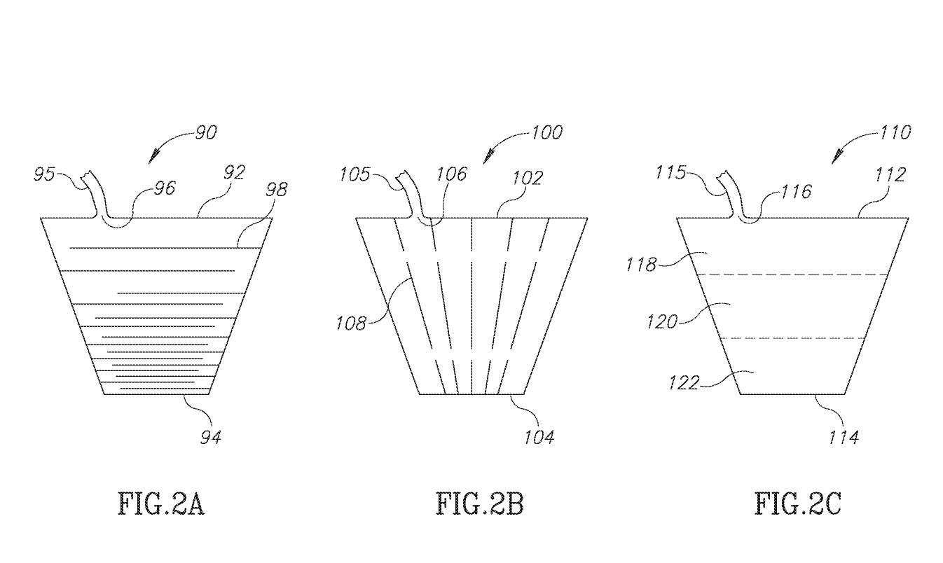

Reference is now made to FIGS. 2A-2C, which show pressure applicators which may be used in IOPC apparatuses according to embodiments of the invention. FIG. 2A shows a cross sectional view of a pressure applicator 90 comprising a fundal end 92, a cervical end 94, an air tube 95, an inlet 96 and internal wall 98. Pressure applicator may positioned between inner and outer layers of an IOPC, with fundal end 92 configured to be placed near uterine fundus and cervical end 94 configured to be placed near cervix. In order to apply pressure on a uterus once pressure applicator is located within an IOPC and secured to a uterus, fluid, for example, air, may enter pressure applicator 90 via air tube 95 and inlet 96 from fundal end 92. At fundal end 92, internal walls are spaced further apart relative to internal wall spacing at cervical end. As a result, pressure applicator 90 expands to a greater extent at its fundal end 92 than at its cervical end 94, thereby applying more pressure to the fundus of a uterus than to the cervical region.

FIG. 2B shows a cross sectional view of a pressure applicator 100 comprising a fundal end 102, a cervical end 104, an air tube 105, an inlet 106 and internal wall 108. Pressure applicator may positioned between inner and outer layers of an IOPC, with fundal end 102 configured to be placed near uterine fundus and cervical end 104 configured to be placed near cervix. In order to apply pressure on a uterus once pressure applicator is located within an IOPC and secured to a uterus, fluid, for example, air, may enter pressure applicator 100 via air tube 105 and inlet 106 from fundal end 102. At fundal end 102, internal walls are spaced further apart relative to internal wall spacing at cervical end. As a result, pressure applicator 100 expands to a greater extent at its fundal end 102 than at its cervical end 104, thereby applying more pressure to the fundus of a uterus than to the cervical region.

FIG. 2C shows a cross sectional view of a pressure applicator 110 comprising a fundal end 112, a cervical end 114, an air tube 115, an inlet 116 and chambers 118, 120 and 122. Pressure applicator may positioned between inner and outer layers of an IOPC, with fundal end 112 configured to be placed near uterine fundus and cervical end 114 configured to be placed near cervix. In order to apply pressure on a uterus once pressure applicator is located within an IOPC and secured to a uterus, fluid, for example, air, may enter pressure applicator 110 via air tube 115 and inlet 116 from fundal end 112. Chambers 118, 120 and 122 are made of materials having different elasticities. Chamber 118 is the most elastic, chamber 120 is less elastic an chamber 122 is the least elastic. Air may flow freely between chambers. As a result, pressure applicator 110 expands to a greater extent at its fundal end 112 than at its cervical end 114, thereby applying more pressure to the fundus of a uterus than to the cervical region.

Embodiments of the invention comprising a soft, flexible outer sleeve of an IOPC are advantageous for treatment of PPH. During PPH, a uterus may be engorged, and may greatly vary in size from patient to patient. IOPC according to embodiments of the invention are easily adjustable and can properly secure a uterus of a variety of sizes. In addition, the structure of IOPC according to embodiments of the invention, in which front and rear sections are attached and only require securing around the sides of the uterus allows for rapid securing of the IOPC to the uterus, thereby minimizing blood loss in PPH.

There is further provided, according to an embodiment of the invention, an apparatus for the reduction of uterine blood flow, the apparatus comprising: a flexible sleeve configured to envelop at least a portion of a uterus; and an adjustable pressure applicator configured to provide pressure on a uterus of a patient. Optionally, the flexible sleeve comprises: a flexible rear section configured to conform to a posterior side of a uterus; a flexible front section configured to conform to an anterior side of a uterus; and a connector for securing the front section to the rear section. Optionally, upon securing the connector, the rear and front sections form a closed sleeve, the closed sleeve having openings configured to allow for the vagina and fallopian tubes to be located on the outside of the sleeve while the uterus is confined within the sleeve. Optionally, the sleeve comprises an outer layer and an inner layer, wherein the inner layer comprises a fabric. Optionally, the pressure applicator is situated between the outer and inner layer. Optionally, the pressure applicator comprises at least one bladder comprising an elastic layer, the bladder adapted to receive and dispel a fluid. Optionally, the pressure applicator further comprises a conduit to convey a fluid from outside of the body of the patient to the bladder. Optionally, the fluid is air. Optionally, the pressure applicator is configured to apply pressure selectively to the fundus of the uterus without applying substantial pressure to the cervix, thereby allowing blood to exit the uterus via the cervix. Optionally, the apparatus further comprises an inflatable bladder adapted to receive and dispel a fluid, the bladder having multiple sections, the sections configured to apply pressure to fundal regions of the uterus and to cervical regions of the uterus. Optionally, each bladder section may be inflated or deflated independently of other bladder sections. Optionally, a bladder section, upon inflation, is configured to apply more pressure to fundal regions of the uterus than to cervical regions of the uterus. Optionally, a bladder section comprises regions having increased elasticity relative to other regions. Optionally, bladder regions having increased elasticity are configured for positioning close to a fundal area of a uterus. Optionally, the apparatus further comprises a pump, flow-connected to the bladder via the conduit. Optionally, the apparatus further comprises at least one sensor configured to detect uterine blood flow. Optionally, the apparatus further comprises a controller configured to receive an input from the sensor and to modulate pressure administered by the pressure applicator, responsive to the input received from the sensor. Optionally, the connector comprises a hook and loop fastener. Optionally, the rear section comprises tabs, the tabs comprising a hook and loop fastener, and the front section comprises a corresponding hook and loop fastener. Optionally, the corresponding hook and loop fastener on the front section covers a majority of the front section. Optionally, the tabs comprise a hook material of a hook and loop fastener and the front section comprises a corresponding loop material.

There is further provided, according to an embodiment of the invention, a method for treatment of post partum hemorrhage in a patient comprising: affixing an apparatus according to claim 1 to a hemorrhaging uterus; applying pressure to the uterus using the apparatus to slow the flow of blood from the uterus; and removing the apparatus from the uterus upon slowing the flow of blood. Optionally, the post partum hemorrhage results from a caesarean section delivery. Optionally, the post partum hemorrhage results from a placenta accreta.

In the description and claims of the present application, each of the verbs, “comprise,” “include” and “have,” and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. An apparatus for the reduction of uterine blood flow, the apparatus comprising:
a flexible sleeve configured to envelop at least a portion of a uterus; and
an adjustable pressure applicator configured to provide pressure on a uterus of a patient.

2. The apparatus according to claim 1 wherein the flexible sleeve comprises:
a flexible rear section configured to conform to a posterior side of a uterus;
a flexible front section configured to conform to an anterior side of a uterus; and
a connector for securing the front section to the rear section;

3. The apparatus according to claim 2, wherein upon securing the connector, the rear and front sections form a closed sleeve, the closed sleeve having openings configured to allow for the vagina and fallopian tubes to be located on the outside of the sleeve while the uterus is confined within the sleeve.

4. The apparatus according to claim 1 wherein the sleeve comprises an outer layer and an inner layer, wherein the inner layer comprises a fabric.

5. The apparatus according to claim 4 wherein the pressure applicator is situated between the outer and inner layer.

6. The apparatus according to claim 1 wherein the pressure applicator comprises at least one bladder comprising an elastic layer, the bladder adapted to receive and dispel a fluid.

7. The apparatus according to claim 6 wherein the pressure applicator further comprises a conduit to convey a fluid from outside of the body of the patient to the bladder.

8. (canceled)

9. The apparatus according to claim 1 wherein the pressure applicator is configured to apply pressure selectively to the fundus of the uterus without applying substantial pressure to the cervix, thereby allowing blood to exit the uterus via the cervix.

10. The apparatus according to claim 9 further comprising an inflatable bladder adapted to receive and dispel a fluid, the bladder having multiple sections, the sections configured to apply pressure to fundal regions of the uterus and to cervical regions of the uterus.

11. The apparatus according to claim 10 wherein each bladder section may be inflated or deflated independently of other bladder sections.

12. The apparatus according to claim 9 wherein a bladder section, upon inflation, is configured to apply more pressure to fundal regions of the uterus than to cervical regions of the uterus.

13. The apparatus according to claim 12 wherein a bladder section comprises regions having increased elasticity relative to other regions.

14. The apparatus according to claim 13 wherein a bladder regions having increased elasticity are configured for positioning close to a fundal area of a uterus.

15. The apparatus according to claim 7 further comprising a pump, flow-connected to the bladder via the conduit.

16-17. (canceled)

18. The apparatus according to claim 2 wherein the connector comprises a hook and loop fastener.

19. The apparatus according to claim 18 wherein the rear section comprises tabs, the tabs comprising a hook and loop fastener, and the front section comprises a corresponding hook and loop fastener.

20. The apparatus according to claim 19 wherein the corresponding hook and loop fastener on the front section covers a majority of the front section.

21. The apparatus according to claim 19 wherein the tabs comprise a hook material of a hook and loop fastener and the front section comprises a corresponding loop material.

22. A method for treatment of post partum hemorrhage in a patient comprising: affixing an apparatus according to claim 1 to a hemorrhaging uterus;
applying pressure to the uterus using the apparatus to slow the flow of blood from the uterus; and
removing the apparatus from the uterus upon slowing the flow of blood.

23. The method according to claim 22 wherein the post partum hemorrhage results from a caesarean section delivery.

24. (canceled)
